(19) United States
(12) Patent Application Publication
(10) Pub. No.: US 2025/0056707 A1
(43) Pub. Date: Feb. 13, 2025

(54) TARGET FOR NEUTRON GENERATION DEVICE, AND PRODUCTION METHOD THEREFOR

(71) Applicants: YAGAMI Co., LTD., NAGOYA-SHI, AICHI (JP); National University Corporation Tokai National Higher Education and Research System, NAGOYA-SHI, AICHI (JP); Metal Technology Co., Ltd., NAKANO-KU, TOKYO (JP)

(72) Inventors: Kazuki TSUCHIDA, NAGOYA-SHI, AICHI (JP); Akira URITANI, NAGOYA-SHI, AICHI (JP); Sachiko YOSHIHASHI, NAGOYA-SHI, AICHI (JP); Shogo HONDA, NAGOYA-SHI, AICHI (JP); Tomohiro YONEMOTO, NAKANO-KU, TOKYO (JP); Yuki HONJO, NAKANO-KU, TOKYO (JP)

(21) Appl. No.: 18/718,994

(22) PCT Filed: Dec. 13, 2022
(86) PCT No.: PCT/JP2022/045945

§ 371 Date: Aug. 9, 2024

(30) Foreign Application Priority Data
Dec. 13, 2021 (JP) 2021-202088

(51) Int. Cl. H05H 6/00; G21K 5/08

(52) U.S. Cl. CPC H05H 6/00; G21K 5/08

(57) ABSTRACT

A target for a neutron generation device includes a target material that is to be irradiated with a proton beam to generate neutrons; a substrate in which a recessed portion that holds the target material is provided; and a metal foil for sealing the target material held in the recessed portion. The target material is a lithium alloy material in which a part or an entire portion of lithium is alloyed. The target material and the substrate are alloyed and joined together. A method for producing a target involves placing a precursor made of metal lithium or a lithium alloy in the recessed portion; alloying the precursor and the substrate with each other to be joined together by heating the substrate and the precursor to melt the precursor, and then, solidifying the precursor; and joining the metal foil on the substrate to which the precursor has been joined.

### TECHNICAL FIELD

The present invention relates to a target for a neutron generation device for generating neutrons by a ⁷Li(p,n)⁷Be reaction, and a production method therefor.

### BACKGROUND ART

Boron neutron capture therapy (BNCT) is a type of radiotherapy to treat cancer. Boron neutron capture therapy is a treatment method that destroys tumor cells by irradiating a boron compound selectively accumulated in the tumor cells with neutrons to generate alpha particles and lithium nuclei by a nuclear reaction represented by ¹⁰B(n,α)⁷Li. Ranges of the alpha particles and lithium nuclei are equivalent to a size of cells. Thus, BNCT makes it possible to selectively destroy only the tumor cells without severely damaging normal cells.

In BNCT, a boron compound containing boron 10(¹⁰B) is administered to a patient in advance, and tumor cells in which the boron compound has been accumulated are irradiated with neutron beams. The lower the energy of neutrons, the larger the reaction cross section is. This feature makes it possible to avoid damaging normal cells, while such high energy is required as to reach deep tissues in the patient. Accordingly, the neutron beams to be used in the treatment are required to be such that intensity of epithermal neutrons is high, a mixing rate of fast neutrons is low, and a rate of a thermal neutron flux to an epithermal neutron flux is low.

The International Atomic Energy Agency (IAEA) has set design target values for neutron beams to be used in boron neutron capture therapy. For example, intensity of epithermal neutrons is recommended to be 1×10⁹ [n·cm⁻²·s⁻¹] or more from a viewpoint of effectively performing the treatment in a short time. Also, the mixing rate of fast neutrons is recommended to be 2×10⁻¹³ [Gy·cm²] or less from a viewpoint of avoiding damage of normal cells.

In recent years, neutron generation devices for use in boron neutron capture therapy have been shifting from research reactors and the like to those using an accelerator. High-speed neutron beams generated by a neutron source are decelerated to or below the epithermal range and then used for treatment. Neutron generation devices using an accelerator generally includes the accelerator, which generates charged particle beams, a target which generates neutron beams in response to being irradiated with the charged particle beams, and a deceleration irradiation device that decelerates the neutron beams generated by the target to irradiate the neutron beams to an irradiation object.

The target holds a target material which functions as a neutron source on a substrate provided with a cooling mechanism. The target material is a material that causes a neutron generating reaction in response to being irradiated with charged particle beams. Here, lithium causing a ⁷Li(p,n)⁷Be reaction, beryllium causing a ⁹Be(p,n)⁹B reaction and a ⁹Be(p,xn) reaction, heavy metals such as tantalum and tungsten causing nuclear spallation reactions, have been investigated as the target material.

Using lithium or beryllium as the target material reduces generation of gamma rays as compared to nuclear spallation reactions by heavy metals. This feature in turn makes shielding for the patient easy and enhances safety of the treatment. Also, using lithium makes it possible to generate neutrons with lower incident proton energy than with beryllium. That is, it is possible not only to reduce the energy of the neutron beams to be generated but also to greatly suppresses generation of secondary radiation.

While a threshold value of the incident proton energy is approximately 2.06 MeV with a ⁹Be(p,n)⁹B reaction, it is approximately 1.88 MeV with a ⁷Li(p,n)⁷Be reaction. Further, when comparing lithium to beryllium, lithium is larger in a macroscopic cross section over the whole range of the incident proton energy. For these reasons, lithium has been regarded as a promising target material suitable for making neutron generation devices less radioactive, smaller, and lighter. Using lithium as a target material reduces unnecessary neutrons and radiation to be emitted from radioactivated devices. Therefore, the use of lithium can greatly reduce radiation exposure of patients and the like.

Non-patent Document 1 describes results of analyzing an impact of blistering on a target obtained by vapor-deposited lithium on a substrate made of copper. Non-patent Document 1 indicates that blistering occurs at a surface of the copper substrate due to hydrogen generated inside the metal as a result of irradiation with proton beams but an impact on a neutron yield is small. Non-patent Document 1 discloses that an amorphous copper layer and lithium-copper alloy layer were formed at an interface between a vapor-deposited lithium film and the copper substrate as a result of irradiation with proton beams.

### PRIOR ART DOCUMENT

### Non-Patent Document

Non-patent Document 1: Timofey Bykov et al., Nuclear Instruments and Methods in Physics Research Section B, Vol. 481, 15 Oct. 2020, p. 62-81

### SUMMARY OF INVENTION

### Technical Problems

In the case of using lithium as a target material for generating neutrons, the target material easily melts since a melting point of lithium is low, i.e., about 180° C. Therefore, the target material easily melts, while energy density of incident protons needs to be high of more than several MW/m². For this reason, the target for the neutron generation device needs a structure to efficiently remove a heat load.

A method of removing heat from a target material held on a substrate includes a method in which a coolant is circulated to and from the substrate to allow heat exchange between the substrate and the coolant. To achieve high cooling efficiency by such a method, the target itself must have good thermal conductivity. Thus, it is necessary not only to use materials with high thermal conductivity for the substrate serving as a component of the target and the like, but also to ensure good heat transfer, i.e., low thermal resistance, between the target material and the substrate and the like.

Also, when a target for generating neutrons is irradiated with protons, the protons stop at a certain depth inside the target. This feature may generate cracks in the target substrate (i.e., cause blistering). Blistering is a phenomenon in which a hydrogen gas causes swelling due to concentrated accumulation of hydrogen inside the target, thus forming a bubble-shaped space inside the substrate material and the like. Blistering as above causes an increase in thermal resistance between the target material and the substrate, and hydrogen embrittlement. Hence, a countermeasure against the blistering is required.

The countermeasure against the blistering includes a method in which the incident proton energy is set high to lengthen a range of the protons to be incident on the target material, and a method in which tantalum having high blistering resistance is used as a backing substrate. However, a problem with the method in which the incident proton energy is set high is that the energy of neutrons generated by the target material is high as well, thus greatly affecting a moderator system and the measures against radiation exposure. Further, a problem with the method that uses a backing substrate is that a material of the substrate is limited, resulting in an obstacle for using a material with high thermal conductivity and a structure with low thermal resistance.

Further, in the case of using lithium as the target material to generate neutrons, the lithium easily reacts with the oxygen, nitrogen, moisture, and so on in the air, and also the lithium may be melt when irradiated with a proton beam. Thus, a structure to seal the lithium is required. In the case of using solid lithium as the target material, the target material, which is held on the substrate, can be sealed by covering it with a metal foil. However, the metal foil in such a structure is subjected to large heat loads and pressures from the target material heated, and therefore good heat transfer and joinability are secured.

For example, if heat transfer between the target material and the metal foil is poor, heat cannot be easily removed from the metal foil with a coolant caused to flow to the substrate side, which may cause burnout of the metal foil. Further, if joinability between the target material and the metal foil is low, a gap is likely to be formed between the target material and the metal foil when thermal expansion and thermal shrinkage occur or an external force is applied. This phenomenon causes an increase in thermal resistance between the target material and the metal foil, and unevenness when the target material is melted.

Non-patent Document 1 discloses that an amorphous copper layer and lithium-copper alloy layer are formed at the interface between a vapor-deposited lithium film which is a target material and a copper substrate, thereby improving an adhesive property between the lithium layer and a surface of the copper substrate. However, with a vapor-deposited film, these layers are formed when irradiating a proton beam, and undergoes erosion of the copper substrate. Thus, the structure is not reliable. Therefore, a structure that achieves good heat transfer and bonding ability between a target material and a substrate and a metal foil at the periphery of the target material has been desired.

Thus, an object of the present invention is to provide a target for a neutron generation device with improved heat transfer and joinability between a target material which generates neutrons via irradiation with a proton beam and members adjacent thereto, and to provide a production method therefor.

### Solution to Problems

A target for a neutron generation device according to the present invention for solving the above problems is a target for a neutron generation device for generating neutrons by a ⁷Li(p,n)⁷Be reaction. The target includes: a target material which generates neutrons via irradiation with a proton beam; a substrate in which a recessed portion to hold the target material is provided; and a metal foil which seals the target material held in the recessed portion. Herein, the target material is a lithium alloy material in which lithium is partly or entirely alloyed, and the target material and the substrate are alloyed with each other to be joined.

Further, a method of producing a target for a neutron generation device according to the present invention is a method of producing a target for a neutron generation device for generating neutrons by a ⁷Li(p,n)⁷Be reaction. That is the method includes: placing a precursor made of metallic lithium or a lithium alloy in a recessed portion provided in a substrate; heating the substrate and the precursor to melt the precursor; and thereafter, alloying the precursor and the substrate with each other to join the precursor and the substrate by letting the melted precursor solidify; and joining a metal foil onto the substrate to which the precursor has been joined.

### Advantageous Effects of Invention

In accordance with the present invention, it is possible to provide a target for a neutron generation device with improved heat transfer and joining ability between a target material which generates neutrons via irradiation with a proton beam and members adjacent thereto, and to provide a production method therefor.

### BRIEF DESCRIPTION OF DRAWINGS

FIG. 1 is a diagram showing a schematic configuration of a neutron generation device including a target.

FIG. 2 is a perspective view showing an example of a neutron generation device target according to an embodiment of the present invention.

FIG. 3 is a vertical cross-sectional view of the neutron generation device target.

FIG. 4 is a horizontal cross-sectional view of the neutron generation device target.

FIG. 5A is a view schematically showing an example of a structure of an alloyed target material.

FIG. 5B is a view schematically showing an example of the structure of the alloyed target material.

FIG. 5C is a view schematically showing an example of the structure of the alloyed target material.

FIG. 6 is a diagram showing an example of a method of producing the neutron generation device target according to the embodiment of the present invention.

FIG. 7 is a diagram showing an energy loss of a proton beam in the neutron generation device target.

FIG. 8 is a set of photographic images showing an exterior of the neutron generation device target using a metal foil with a Cu coating before and after being irradiated with an electron beam.

FIG. 9 is a set of infrared photographic images of the neutron generation device target using the metal foil with a Cu coating during the electron beam irradiation.

FIG. 10 is a diagram showing a temporal change in a current value and a temporal change in degree of vacuum during the electron beam irradiation of the neutron generation device target using the metal foil with a Cu coating.

FIG. 11 is a diagram showing the temporal change in density of heat input during the electron beam irradiation of the neutron generation device target using the metal foil with a Cu coating.

FIG. 12 is a set of photographic images showing the exterior of a neutron generation device target using a metal foil with no Cu coating before and after being irradiated with an electron beam.

FIG. 13 is a set of infrared photographic images of the neutron generation device target using the metal foil with no Cu coating during the electron beam irradiation.

FIG. 14 is a diagram showing the temporal change in the current value and the temporal change in degree of vacuum during the electron beam irradiation of the neutron generation device target using the metal foil with no Cu coating.

FIG. 15 is a diagram showing the temporal change in the density of heat input during the electron beam irradiation of the neutron generation device target using the metal foil with no Cu coating.

FIG. 16 is a set of photographic images showing the exterior of a neutron generation device target using the metal foil with the Cu coating before and after being irradiated with the proton beam.

FIG. 17 is a set of infrared photographic images of the neutron generation device target using the metal foil with the Cu coating during the proton beam irradiation.

### DESCRIPTION OF EMBODIMENTS

Hereinafter, a target for a neutron generation device according to an embodiment of the present invention and a production method therefor will be described with reference to the drawings.

### <Neutron Generation Device>

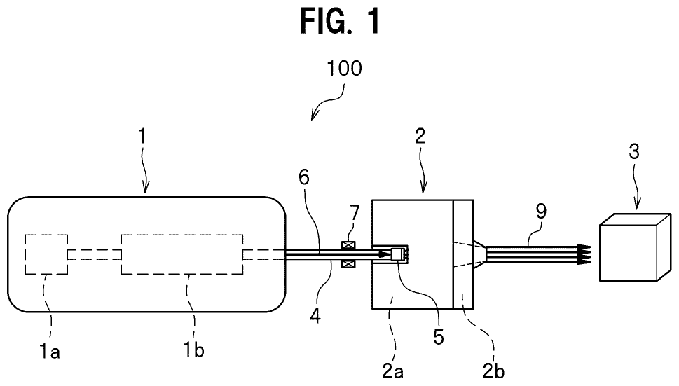

FIG. 1 is a diagram showing a schematic configuration of a neutron generation device including a target.

As shown in FIG. 1, a neutron generation device 100 includes a proton beam generation device 1, a neutron deceleration irradiation device 2, a channel 4, and a neutron generation device target 5. Reference sign 3 denotes an irradiation object, reference sign 6 denotes a proton beam, reference sign 7 denotes a magnetic focusing lens, and reference sign 9 denotes neutron beams.

The neutron generation device 100 is a device that generates neutron beams with controlled neutron characteristics. The neutron generation device 100 uses solid lithium to generate neutrons. Further, the neutron generation device 100 uses an accelerator as a beam source for charged particle beams that causes a nuclear reaction. The neutron generation device 100 generates neutrons by a nuclear reaction represented by ⁷Li(p,n)⁷Be and irradiates a targeted irradiation object 3 with neutron beams controlled to be within an energy band of epithermal neutrons.

The neutron generation device 100 is usable as a neutron source, for example, in a boron neutron capture therapy (BNCT). In BNCT applications, the proton beam generation device 1 and the neutron deceleration irradiation device 2 can be placed inside a hospital, a therapeutic facility, or the like, for example. The neutron generation device 100 can be used in treatment of a malignancy patient with cancer in the head, neck, or the like.

When the neutron generation device 100 is in operation, the proton beam generation device 1 generates the proton beam 6 with a predetermined energy. The proton beam 6 is irradiated to the target 5 through the channel 4. In response to incidence of the proton beam 6 with the predetermined energy on the target 5, the target 6 causes a nuclear reaction represented by ⁷Li(p,n)⁷Be, thereby generating neutron beams. The neutron deceleration irradiation device 2 decelerates the neutrons emitted by the target 5 to a predetermined energy band to emit shaped neutron beams 9.

The neutron beams 9 emitted from the neutron deceleration irradiation device 2 are irradiated to the targeted irradiation object 3. The irradiation object 3 is a diseased tissue or the like of the malignancy patient. A boron compound accumulated in a targeted object such as tumor cells is administered in advance to a patient who is to receive BNCT treatment. When cells in which boron 10(¹⁰B) has been accumulated are irradiated with the neutron beams 9, alpha particles and lithium nuclei are generated by a nuclear reaction represented by ¹⁰B(n,α)⁷Li. The irradiation of these nuclear species can damage the tumor cells in the diseased tissue or the like to treat the malignancy or the like.

In BNCT applications, an epithermal neutron flux of the neutron beams 9 irradiated to the irradiation object 3 is recommended to be 1×10⁹ [n/cm²/s] or more from the viewpoint of effectively performing the treatment in a short time. Further, a mixing rate of fast neutrons included in the neutron beams 9 is recommended to be 2×10⁻¹³ [Gy/cm²] or less from the viewpoint of avoiding damage of normal cells. Moreover, it is also necessary to prevent inclusion of fast neutron beams and gamma rays in the neutron beams 9 emitted from the neutron deceleration irradiation device 2.

For this reason, the neutron deceleration irradiation device 2 uses an appropriate type of moderation material and an appropriately configured moderator system to decelerate the neutrons generated by the target 5 to the energy band of epithermal neutrons. The neutron deceleration irradiation device 2 also uses appropriate types of moderation material, shielding material, and the like and appropriately configured moderator system, collimator, and the like to shield components such as fast neutron beams and gamma rays included in the neutron beam generated by the target 5, and irradiates neutron beams with necessary neutron characteristics toward the irradiation object 3.

As shown in FIG. 1, the proton beam generation device 1 includes an ion source 1a that generates protons and an accelerator 1b that accelerates the protons. As the ion source 1a, for example, an electron cyclotron resonance (ECR) ion source is used. As the accelerator 1b, for example, an electrostatic accelerator is used. Alternatively, a linear accelerator or the like may be used.

The ECR ion source introduces a hydrogen gas into a chamber under a strong magnetic field and applies heigh frequency waves to induce electron cyclotron resonance and thereby generate a high-density hydrogen plasma. Hydrogen ions (¹H+) generated inside the chamber are selected from among other ionic species under a magnetic field created by a multipolar magnetic mirror, and are drawn out of the chamber by an electrode. Advantageously, the ECR ion source is capable of stably operating for a long time since it performs electrodeless discharge.

The electrostatic accelerator applies a high DC voltage between electrodes to accelerate the charged particles under a constant electrostatic field. The electrostatic accelerator can generate a continuous proton beam 6. As the electrostatic accelerator, for example, a Dynamitron accelerator, a Cockcroft-Walton accelerator, a Van de Graaff accelerator, or the like can be used. The linear accelerator applies an AC electric field inside a tubular electrode to accelerate the charged particles inside a radio-frequency AC electric field. Examples of the linear accelerator include a radio-frequency quadrupole (RFQ) linac and the like.

The channel 4 forms a high-vacuum path that connects the proton beam generation device 1 and the target 5 and guides protons generated by the proton beam generation device 1 to the target 5. The magnetic focusing lens 7 for keeping the proton beam 6 from spreading in the width direction is placed at an intermediate portion of the channel 4. A collimator for converging the proton beam 6 is placed at the terminal end of the channel 4.

The magnetic focusing lens 7 can be formed of, for example, a plurality of quadrupole electromagnets. The quadrupole electromagnets can be disposed with their respective poles inverted on the channel 4 along the direction in which the proton beam 6 is irradiated. The collimator can be provided, for example, in a shape having a through-hole corresponding to a predetermined beam diameter. The collimator with such a shape shields the proton beam 6 directed away from the irradiation direction to narrow the proton beam 6 to be irradiated to the target 5.

Note that the channel 4 is not limited to a straight path as shown in FIG. 1. The channel 4 can be provided in a form of path of any shape having a curved portion(s) according to where the neutron generation device 100 is placed or the like. At the curved portion (s) of the channel 4, a bending magnet that deflects the proton beam 6 or the like can be disposed. For example, it is possible to place the proton beam generation device 1 in a machine room of a hospital, a therapeutic facility, or the like and place the neutron deceleration irradiation device 2 and the target 5 in a treatment room and irradiate the neutron beams 9 to the irradiation object 3 in the treatment room.

The target 5 is supported on a target holder disposed at the terminal end of the channel 4. The target 5 holds a solid target material, and generates neutrons by a nuclear reaction represented by ⁷Li(p,n)⁷Be when irradiated with the proton beam 6. The target 5 is subjected to a heat load due to the heat input by the proton beam 6. For this reason, the target 5 and the target holder are provided with a cooling mechanism. A mechanism that removes heat by heat exchange through circulation of a coolant to and from the target 5 can be included in the cooling mechanism.

A threshold value of the energy for generating neutrons in a nuclear reaction represented by ⁷Li(p,n)⁷Be is approximately 1.88 MeV. Here, if the energy of the protons to be incident on the target 5 is excessively high, high-energy neutrons are generated. Then, to decelerate the high-energy neutrons, a large moderator and advanced shielding to avoid radiation exposure of a patient will be necessary. For this reason, the proton beam generation device 1 generates a proton beam 6 having an energy that is more than or equal to this threshold value and is so low as to prevent generation of high-energy neutrons.

The energy of the proton beam 6 generated by the proton beam generation device 1 is preferably 3.0 MeV or less and more preferably 2.9 MeV or less. Also, the energy is preferably 2.5 MeV or more and more preferably 2.7 MeV or more. Further, the current value used for the proton beam generation device 1 is 10 mA or more and 100 mA or less, and is preferably 10 mA or more 20 mA or less from the viewpoint of reducing the heat load on the target 5.

As shown in FIG. 1, the neutron deceleration irradiation device 2 includes a moderator 2a that decelerates neutrons and a collimator 2b that shapes neutron beams. The neutron deceleration irradiation device 2 decelerates the neutrons generated by the target 5 mainly to the energy band of epithermal neutrons, and converges the neutrons spreading in all directions from the target 5 into a single irradiation direction and emits the neutron beams 9 thus shaped toward the irradiation object 3.

Note that the shape and structure of the neutron deceleration irradiation device 2 are schematically shown in FIG. 1. The neutron deceleration irradiation device 2 can be provided in any shape and structure as long as they do not impair the function of decelerating neutrons, the function of shaping or irradiating neutron beams, and the like. The moderator 2a and the collimator 2b are formed by combining a moderation material that decelerates neutrons, a reflection material that reflects neutrons, an absorption material that absorbs neutrons, a shielding material that shields neutrons and gamma rays, and so on in an appropriate arrangement.

The moderator 2a can be disposed so as to surround the lateral sides and the rear side (i.e., the downstream side in the irradiation direction of the proton beam 6) of the target 5. The moderator 2a decelerates fast neutrons and the like generated by the target 5 mainly to the energy band of epithermal neutrons. Further, while preventing leakage of the neutrons generated by the target 5, the moderator 2a reflects and scatters the neutrons spreading from the target 5 toward the collimator 2b.

As shown in FIG. 1, in the moderator 2a, a path through which the proton beam 6 is incident on the target 5 is provided in front of the target 5 (i.e., the upstream side in the application direction of the proton beam 6). No moderation material or reflection material is disposed in the incidence path for the proton beam 6, and the target holder that holds the target 5 is disposed at the terminal end of that path. A moderation material that decelerates neutrons and a shielding material that shields thermal neutrons and gamma rays are disposed behind the target 5. An amplification material that amplifies neutrons may be disposed between the target 5 and the moderator 2a.

Further, the moderation material is also disposed on lateral sides of the target 5 so as to surround the target 5. A reflection material that reflects neutrons and an absorption material that absorbs neutrons are disposed on the outer side of the moderation material excluding the incidence path for the proton beam 6 and a space behind the target 5. The moderation material disposed on the lateral sides of the target 5 is preferably disposed such that its upstream end in the application direction of the proton beam 6 is located upstream of the target 5. Such an arrangement can decelerate neutrons spreading toward the upstream side and also reflect and scatter them toward the collimator 2b. Accordingly, an epithermal neutron flux emitted from the neutron deceleration irradiation device 2 can be increased.

Magnesium fluoride (MgF₂) or calcium fluoride (CaF₂) is preferably used as a moderation material. Monocrystalline magnesium fluoride or calcium fluoride or sintered magnesium fluoride or calcium fluoride obtained by sintering single crystals can be used. Magnesium fluoride and calcium fluoride more efficiently decelerate neutrons when the energy of the protons incident on the target 5 is 10 MeV or less. For this reason, using magnesium fluoride or calcium fluoride makes it possible to efficiently decelerate the neutrons generated by the target 5 to the energy band of epithermal neutrons and absorb most of the fast neutrons.

As a reflection material, for example, lead (Pb), graphite (C), iron (Fe), beryllium (Be), bismuth (Bi), or the like can be used. Lead or a lead alloy such as a lead-tin alloy, a lead-antimony alloy, or a lead-bismuth alloy is preferable as the reflection material for their features such as a large neutron scattering cross-section, low epithermal neutron absorption, and a high gamma ray shielding ability.

As an absorption material, for example, a boron compound such as boron-polyethylene, lithium fluoride-polyethylene, a paraffin, or boron carbide or the like can be used.

As a shielding material, for example, cadmium (Cd), indium (In), hafnium (Hf), gadolinium (Gd), bismuth (Bi), iron (Fe), lead (Pb), lead fluoride (PbF₂), a boron compound such as boron carbide, a paraffin, water, lithium fluoride-polyethylene, boron-polyethylene, or the like can be used.

The collimator 2b is disposed on the rear side (i.e., a downstream side in the irradiation direction of the proton beam 6) of the moderator 2a. The collimator 2b focuses the neutron beam decelerated by the moderator 2a to a predetermined beam diameter while also shielding the thermal neutrons and gamma rays included in the neutron beams 9 emitted from the neutron deceleration irradiation device 2 and the like to prevent these radiations from being irradiated toward the irradiation object 3.

As shown in FIG. 1, an emitting path for the neutron beams 9 from the target 5 is provided in a center of the collimator 2b. No moderation material is disposed in the emitting path for the neutron beams 9, and the emitting path is formed of an air layer and a shield made of the shielding material to allow the neutron beams 9 to converge and travel straight ahead. A reflection material and the like are disposed on lateral sides of the emitting path for the neutron beams 9 so as to surround the emitting path. The reflection material disposed on the lateral sides of the emitting path for the neutron beams 9 can be provided in a tapered shape that narrows toward the rear side of the collimator 2b. An absorption material that absorbs neutrons and a shielding material that shields neutrons and gamma rays are preferably disposed at a rear portion of the collimator 2b from the viewpoint of reducing radiation exposure.

As shown in FIG. 1, an extension collimator can be provided on a rear side of the collimator 2b so as to protrude to the outside of the device. The extension collimator protrudes rearward (i.e., toward a downstream side in the irradiation direction of the proton beam 6) from a peripheral edge of the emitting path for the neutron beams 9, and defines a tapered conical shape narrowing toward the rear side. The extension collimator is formed by combining a reflection material, an absorption material, a shielding material, and the like in an appropriate arrangement.

Like the collimator 2b, an emitting path for the neutron beams 9 is provided in a center of the extension collimator. The emitting path for the neutron beams 9 can be formed of an air layer and a shield made of the shielding material. A reflection material and the like are disposed on lateral sides of the emitting path for the neutron beams 9 so as to surround the emitting path. The reflection material disposed on the lateral sides of the emitting path for the neutron beams 9 can be provided in a tapered shape that narrows toward a rear side of the extension collimator. The absorption material can be disposed, for example, on the outer side of the reflection material.

In the case of BNCT or the like on a head or neck, providing such an extension collimator makes it possible to irradiate a diseased tissue to be treated more directly with the neutron beams 9. This procedure reduces interference between a patient and the device, and therefore enables precise irradiation of the diseased tissue with the predetermined neutron beams 9 without forcing the patient to assume a difficult posture. Accordingly, the treatment can be done with strong epithermal neutron beams in a short time, thereby reducing radiation exposure. Note that the placement of the extension collimator may be omitted.

Next, details of the neutron generation device target 5 used as a neutron source for the neutron generation device 100 will be described with reference to drawings. One characteristic feature of this target 5 is to use a lithium alloy material obtained by partly or entirely alloying lithium as a target material that generates neutrons. The target material is alloyed by diffusion of atoms with a substrate that holds the target material and the like to be joined thereto.

### <Neutron Generation Device Target>

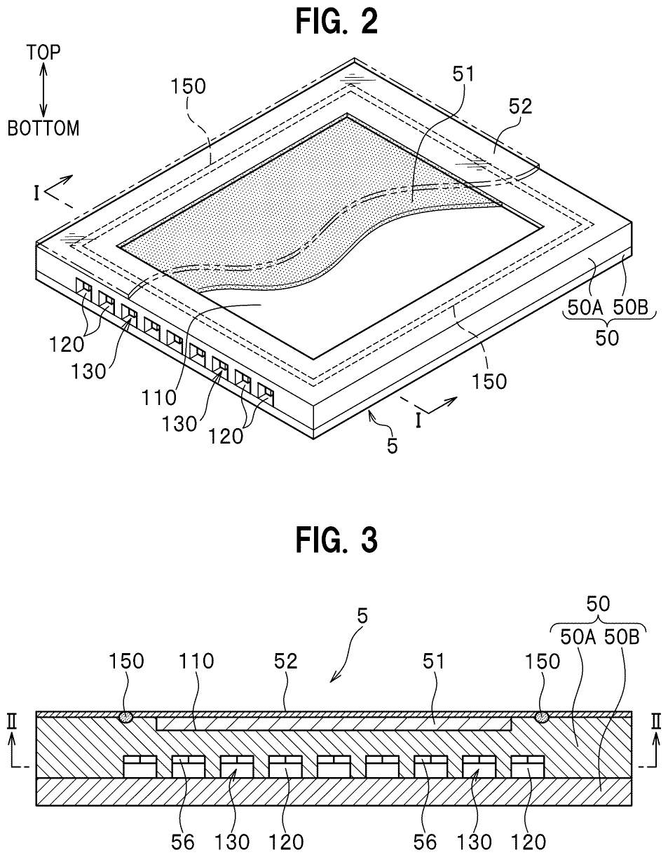

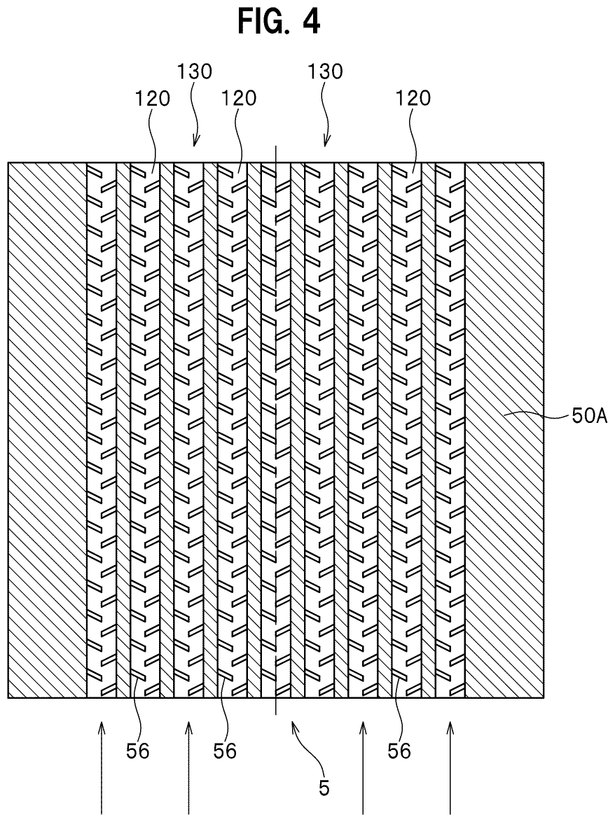

FIG. 2 is a perspective view showing an example of the neutron generation device target according to the embodiment of the present invention. FIG. 3 is a vertical cross-sectional view of the neutron generation device target. FIG. 4 is a horizontal cross-sectional view of the neutron generation device target. FIG. 3 shows a cross-sectional view taken along the I-I line of FIG. 2. FIG. 4 shows a cross-sectional view taken along the II-II line of FIG. 3. FIGS. 2, 3, and 4 show an example in which the neutron generation device target 5 to be used as a neutron source for the neutron generation device 100 is exemplarily provided in a rectangular shape in a plan view.

As shown in FIGS. 2, 3, and 4, the neutron generation device target 5 includes a substrate 50, a target material 51, and a metal foil 52. A length and width of the neutron generation device target 5 can be set, for example, to 5 mm or more and 15 mm or less. However, the size and shape of the neutron generation device target 5 are not particularly limited as long as they can generate predetermined neutrons.

The substrate 50 serves as a body of the target 5 and holds the target material 51. In FIGS. 2, 3, and 4, the substrate 50 is provided in a rectangular shape in a plan view and formed by stacking a holding plate 50A and a back plate 50B. The holding plate 50A and the back plate 50B are provided in a rectangular shape in a plan view and have the same length and width. The target material 51 is held on a main surface of the holding plate 50A on a front side thereof.

In the neutron generation device 100, the substrate 50 is mounted to the predetermined target holder to fix the target 5 at a position irradiated with the proton beam 6. The target holder can be disposed such that it is integrally coupled to the collimator at the terminal end of the channel 4. The target holder can be equipped with a cooling mechanism to remove heat from the target material 51 on which the proton beam 6 is incident and the like. Examples of the cooling mechanism include a mechanism that circulates a coolant for cooling the target material 51 and the like by heat exchange to and from the target 5.

In the neutron generation device 100, the holding plate 50A is disposed on a side of the target 5 on which the proton beam 6 is incident, i.e., the channel 4 side in FIG. 1. On the other hand, the back plate 50B is disposed on a side of the target 5 from which the neutron beams emit, i.e., the moderator 2a side in FIG. 1. The proton beam 6, which causes a nuclear reaction represented by ⁷Li(p,n)⁷Be, is irradiated to around a center of the holding plate 50A.

As shown in FIG. 2, the holding plate 50A is formed in such a shape that the main surface on the front side is thinned except for an outer peripheral portion, and has a recessed portion 110 inside the frame-shaped outer peripheral portion. The recessed portion 110 assumes a rectangular shape in a plan view and forms such a cuboidal space that a thickness thereof is uniformly reduced. The recessed portion 110 is a portion to hold the target material 51 in a tightly sealed manner.

The structure to hold the target material 51 in the recessed portion 110 ensures that the target material 51 in the recessed portion 110 is tightly sealed with the metal foil 52. Accordingly, lithium in the target material 51 is properly prevented from reacting with the oxygen, nitrogen, moisture, and so on in the air. Moreover, even if the target material 51 melts due to heat input by the proton beam 6, the target material 51 can be prevented from leaking.

Incidentally, in FIG. 2, illustration of part of the target material 51 in the recessed portion 110 is omitted. However, when the target 5 is used, the target material 51 is held inside the entire recessed portion 110 with substantially no gap therebetween. The structure with substantially no gap prevents the target material 51 from becoming uneven inside the recessed portion 110 when it melts due to heat input by the proton beam 6. This feature enables repetitive use of the target 5 and stabilization of the radiation quality of the neutron beams 9 generated by the target 5.

As shown in FIGS. 3 and 4, a main surface of the holding plate 50A on the back side is thinned in a shape of grooves, and has a plurality of straight grooves 120 on a back side of the recessed portion 110. Each straight groove 120 assumes a long and narrow rectangular shape in a plan view of the holding plate 50A. Further, at each straight groove 120, a thickness thereof is reduced to a predetermined thickness across a predetermined width, and the straight groove 120 assumes a rectangular in a vertical cross-sectional view. The straight grooves 120 are arranged side by side in parallel to each other at predetermined intervals and provided so as to penetrate through the back surface of the holding plate 50A from one end to the other end.

The holding plate 50A and a back plate 50B thereof are each preferably made of copper or a copper alloy and particularly preferably made of copper. When the holding plate 50A and the back plate 50B are each made of such a material with high thermal conductivity, it is possible to efficiently remove heat from the target material 51 and the like subjected to a heat load due to the heat input by the proton beam 6 by causing a coolant to flow through cooling channels 130 formed by the straight grooves 120. Moreover, the material has excellent mechanical strength and workability, and therefore a high-strength substrate 50 molded in a predetermined shape can be obtained at a relatively low cost. Furthermore, the material is less likely to produce gamma rays when irradiated with the proton beam 6, and can therefore reduce secondary radiation.

Note that increasing energy of the proton beam 6 irradiated to the target 5 makes it easier for the proton beam 6 to pass through the target material 51 and therefore reduces generation of gamma rays inside the target material 51. However, under such an irradiation condition, there is a risk of blistering on the substrate 50 on the rear side of the target material 51. The blistering may cause, for example, detachment of the target material 51 from the holding plate 50A and an increase in the thermal resistance between the target material 51 and the holding plate 50A. To avoid the blistering, it is usually required to use tantalum, which has high blistering resistance, or the like as a backing substrate.

In contrast, it is possible to prevent the blistering without using a material with high blistering resistance as a backing substrate by making the energy of the proton beam 6 to be irradiated to the target 5 slightly lower than the conventional level, thickening the target material 51 to about 250 μm or more, and setting such an irradiation condition as to hold the end of the range of the proton beam 6 inside the target material 51. Since there is no need to use a material that has high blistering resistance but low thermal conductivity, the holding plate 50A and the back plate 50B can be made only of a material with high thermal conductivity, such as copper or a copper alloy. Using only a material with high thermal conductivity, such as copper or a copper alloy, improves the efficiency of cooling the target material 51 and the like as compared to conventional members.

As shown in FIGS. 2 and 3, the holding plate 50A and the back plate 50B are joined in a stacked state. As for the joining method, for example, various methods including metallurgical joining methods such as pressure welding, diffusion welding, welding, and brazing, mechanical joining methods using bolts, screws, a clamp, and the like, and so on are usable. While the holding plate 50A and the back plate 50B are joined so as to ensure watertightness at their joining surfaces around the straight grooves 120, a sealing material or the like may be used as necessary. The holding plate 50A can be processed by milling, electrical discharge machining, etching, or the like.

As shown in FIGS. 2 and 3, when the holding plate 50A and the back plate 50B are stacked, the straight grooves 120 provided in the holding plate 50A form cooling channels 130. The cooling channels 130 have a rectangular shape in a vertical cross-sectional view of the substrate 50 and are formed to be arranged side by side in parallel to one another at predetermined intervals. Further, the cooling channels 130 are formed as structures penetrating through the substrate 50 from one end surface to the other end surface on the opposite side. The cooling channels 130 serve as flow paths through which the coolant for cooling the target material 51, the substrate 50, and the like is caused to flow.

The cooling channels 130 are connected to a coolant supply path for supplying the coolant to the substrate 50 and a coolant discharge path for discharging the coolant from the substrate 50 at the one end surface of the substrate 50 and the other end surface on the opposite side, respectively. The coolant supply path and the coolant discharge path can be formed with manifolds attached to the substrate 50, channels provided in the target holder, pipes such as tubes or hoses, or the like.

For example, water can be used as the coolant to be caused to flow through the cooling channels 130. A temperature of the coolant at the inlets of the cooling channels 130 can be, for example, a normal temperature (e.g., 20±15° C.). Further, an average flow speed of the coolant in the cooling channels 130 is, for example, 2 m/s or more and preferably 5 m/s or more. The coolant can be caused to flow as parallel flows through the cooling channels 130 arranged side by side while the target 5 is irradiated with the proton beam 6. The arrows in FIG. 4 indicate a flow direction of the coolant.

As shown in FIG. 4, the cooling channels 130 can be provided as structures having a plurality of ribs 56 protruding from a wall surface on the side where the target material 51 is held, i.e., a main surface of the holding plate 50A on the back side. The ribs 56 are provided on both the left and right sides of the cooling channels 130 with respect to respective center axes along their longitudinal direction (i.e., indicated by the dashed line in the figure). The ribs 56 are arrayed at predetermined intervals along the longitudinal direction of the cooling channels 130 on each of both the left and right sides with respect to the central axes. The intervals between the ribs 56 are the same on both the left and right sides.

The ribs 56 extend from the wall surfaces on both the left and right sides of the cooling channels 130 with respect to the center axes to the positions of the center axes. As shown in FIG. 3, in a vertical cross-sectional view of the cooling channels 130, the ribs 56 extend from the wall on one side with respect to the center axes to the wall on the other side substantially with no gap therebetween. The ribs 56 protrude from the main surface of the holding plate 50A on the back side, in which the straight grooves 120 are formed, to a height of about ⅕ of the width of the cooling channels 130 in the left-right direction.

As shown in FIG. 4, the ribs 56 assume parallelogram shapes in a plan view of the holding plate 50A. The ribs 56 on the left and right extend obliquely so as to be located farther on the downstream side in the flow direction of the coolant with separation from the position of the center axis of the cooling channel 130 toward the outer side in the left-right direction. The ribs 56 on the left and right, which are arrayed at given intervals, are arrayed so as to be staggered along the flow direction of the coolant across the center axis of the cooling channel 130.

Providing such ribs 56 increases surface areas of the cooling channels 130 and also makes the flow of the coolant turbulent, and therefore enhances the efficiency of cooling the target material 51 and the like with the coolant. The flow of the coolant collides with and gets separated from a rib 56 and then contacts the next rib 56 disposed on the downstream side again. Further, upon colliding with a rib 56, the flow of the coolant moves into the target material 51 side, so that a secondary circulating flow having a swirling shape with a rotation axis perpendicular to the center axis of the cooling channel 130 is generated between the ribs 56. This phenomenon makes the flow of the coolant more turbulent and promotes destruction of a vapor film with the collision of the coolant, so that an effect of greatly improving the heat transfer coefficient is achieved.

In particular, the ribs 56 are inclined so as to be located farther on the downstream side in the flow direction of the coolant as it extends toward the outer side in the left-right direction. This structure proactively transforms the flow of the coolant that has collided with a rib 56 into a flow in the left-right direction, so that a swirling flow and a shear flow flowing in the left-right direction are developed. Further, the ribs 56 on the left and right are arrayed to be staggered along the flow direction of the coolant. This configuration allows the swirling flow and shear flow flowing in the left-right direction to be alternately developed in an opposite direction. Thus, with these structures, the flow of the coolant is made greatly turbulent, thereby enhancing the cooling efficiency.

Micropits in a form of minute recesses are preferably formed in wall surfaces of the cooling channels 130 closer to the target material 51. The micropits can be provided as tapered holes substantially in a form of inverted conical depressions in the wall surfaces of the cooling channels 130. Bases of such tapered holes may be circular bases, U-shaped bases, or the like. The micropits, for example, can be formed by performing laser processing on the wall surfaces of the cooling channels 130 on the target material 51 side, i.e., the main surface of the holding plate 50A on the back side.

The micropits can be provided in the wall surfaces of the cooling channels 130 on the target material 51 side between the ribs 56 arrayed along the longitudinal direction of the cooling channels 130. It is preferable to provide many micropits in a suitable matrix pattern, such as a lattice pattern or a staggered pattern. In particular, on the wall surfaces of the cooling channels 130 on the target material 51 side, the microbits are preferably provided in halves of the regions between the ribs 56 that are located on the upstream side along the flow of the coolant.

The micropits thus provided function as boiling nuclei for the coolant. As the target material 51 is heated by the heat input by the proton beam 6, a temperature of the coolant flowing through the cooling channels 130 rises and a vapor is generated. The vapor generated on the target material 51 side is trapped and grows in the micropits and is released from the micropits in a form of expanded bubbles. This phenomenon promotes cooling by nucleate boiling and thus greatly enhances efficiency of the heat removal with the coolant.

As shown in FIGS. 2 and 3, the target material 51 is held in the recessed portion 110 provided in the holding plate 50A with substantially no gap therebetween. As the target material 51, a solid lithium alloy material is included which is obtained by partly or entirely alloying lithium and joined to the surrounding member by alloying. The neutron generation device 100 irradiates such a solid lithium alloy material with the proton beam 6 to cause the material to generate neutrons by a nuclear reaction represented by ⁷Li(p,n)⁷Be.

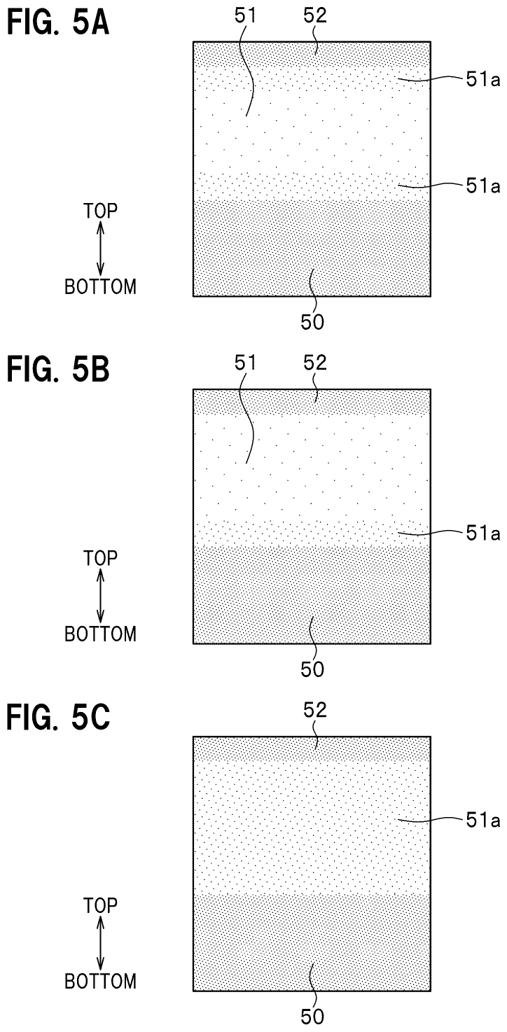

FIGS. 5A, 5B, and 5C are views schematically showing examples of a structure of an alloyed target material. FIGS. 5A, 5B, and 5C are vertical cross-sectional views of a main portion of the target material 51 and schematically shows each constituent layer of the target 5 based on the concentration of the alloying element.

In FIGS. 5A, 5B, and 5C, reference sign 51a denotes an alloy layer in which lithium and another alloying element are alloyed. The regions with light shading are regions where the concentration of lithium is high and the concentration of the alloying element other than lithium is low. The regions with darker shading are regions where the concentration of the alloying element other than lithium is high.

The target material 51 may be a structure in which lithium is partly alloyed as shown in FIGS. 5A and 5B, or a structure in which lithium is entirely alloyed as shown in FIG. 5C. Further, both the side near the substrate 50 and the side near the metal foil 52 may be alloyed as shown in FIG. 5A, or only the side near the substrate 50 may be alloyed as shown in FIG. 5B.

In FIG. 5A, a portion of the target material 51 near the substrate 50 and a portion of the target material 51 near the metal foil 52 are made of a lithium alloy in which the concentration of the alloying element is high, and the remaining portion of the target material 51 separated from the substrate 50 and the metal foil 52 is made of a lithium metal in which the concentration of the alloying element is low. The target material 51 and the substrate 50 or the target material 51 and the metal foil 52 are integrally joined to each other with an alloy layer 51a therebetween in which the atoms are interdiffused.

In FIG. 5B, a portion of the target material 51 near the substrate 50 is made of a lithium alloy in which the concentration of the alloying element is high, and the remaining portion of the target material 51 separated from the substrate 50 is made of a lithium metal in which the concentration of the alloying element is low. The target material 51 and the substrate 50 are integrally joined to each other with an alloy layer 51a therebetween in which the atoms are interdiffused. The target material 51 and the metal foil 52 are provided to be in tight contact with each other at the interface therebetween.

In FIG. 5C, the entire target material 51 is made of a lithium alloy in which the concentration of the alloying element is high. An alloy layer 51a of the target material 51 and the substrate 50, or the alloy layer 51a of the target material 51 and the metal foil 52 are integrally joined to each other such that the atoms are interdiffused. The alloying element contained in the alloy layer 51a may be diffused from the substrate 50 or the metal foil 52, or contained in the target material 51 in advance.

The alloyed target material 51 can be formed by a method in which the precursor of the target material 51, such as lithium metal, and the substrate 50 and the metal foil 52 are brought into tight contact with one another, and thereafter these are subjected to heat treatment to interdiffuse their atoms. The alloying element other than lithium metal can be added to a precursor of the target material 51, such as lithium metal, in advance.

Examples include: a method in which, with the substrate 50 and the metal foil 52 as materials, the precursor of the target material 51, the substrate 50, and the metal foil 52 are brought into tight contact with one another and subjected to heat treatment using a metallic material that can be alloyed with lithium; a method in which a metal layer is formed between the target material 51 and the metal foil 52, and the precursor of the target material 51 and the metal layer in tight contact with each other are subjected to heat treatment; a method in which a metal foil is placed between the target material 51 and the substrate 50, and the precursor of the target material 51 and the metal foil are brought into tight contact with each other and subjected to heat treatment; and the like.

The alloyed target material 51 is preferably a lithium alloy containing lithium (Li) as a main component and one or more of copper (Cu), aluminum (Al), magnesium (Mg), and zinc (Zn). Specific examples of the lithium alloy include a lithium-copper alloy, a lithium-aluminum alloy, a lithium-magnesium alloy, a lithium-zinc alloy, a multielement alloy containing three or more of these elements, and so on. The content of Cu is preferably 20 at % or less. A content of Al is preferably 45 at % or less. A content of Mg is preferably 70 at % or less.

As shown in FIGS. 2 and 3, the metal foil 52 is provided so as to tightly seal the target material 51 held in the recessed portion 110. In each figure, a vertical width and horizontal width of the metal foil 52 are set to be larger than a vertical width and horizontal width of the recessed portion 110, and the metal foil 52 covers the entire recessed portion 110.

An outer peripheral portion of the metal foil 52 may be joined to an outer peripheral portion of the main surface of the holding plate 50A on the front side thereof by pressure welding or the like, or joined with a joint portion 150 provided by working, or joined by pressure welding or the like and then joined by providing the joint portion 150 by working. In the case of joining by interdiffusion of atoms by pressure welding or the like, a metal layer is preferably formed between the target material 51 and the metal foil 52. Note that, in FIG. 2, the joint portion 150 is formed along the entire outer peripheral portion of the metal foil 52. The joint portion 150 can be formed by various methods such as electron beam welding, laser welding, pressure welding, diffusion welding, and clinching. By providing the joint portion 150, it is possible to close any gaps between the holding plate 50A and the metal foil 52, and ensure air tightness and liquid tightness inside the recessed portion 110.

A center portion of the metal foil 52 may be alloyed with the target material 51 to be joined thereto or not joined to the target material 51 as long as the center portion of the metal foil 52 is in tight contact with the target material 51. In the case where the center portion of the metal foil 52 is not joined to the target material 51, an outer peripheral portion of the metal foil 52 needs to be joined to the holding plate 50A. Here, from the viewpoint of ensuring sufficient heat transfer and joinability between the members, it is preferable to alloy the center portion of the metal foil 52 with the target material 51 to be joined, and also join the outer peripheral portion of the metal foil 52 to the holding plate 50A.

As the metal foil 52, a foil made of a metal that does not easily react with the material of the target material 5, which is lithium or the like, and has high proton permeability and heat resistance. As the metal foil 52, titanium, a titanium alloy, beryllium, a beryllium alloy, for example, stainless steel, or the like can be used. These metals can air-tightly and liquid-tightly seal the target material 51 and also prevent a loss of the proton beam 6 irradiated to the target material 51 and heat generation of the metal foil 52 by the proton beam 6.

While a thickness of the metal foil 52 depends on energy of the proton beam 6 to be irradiated to the target 5 and the like, the thickness is preferably 4 μm or more and 20 μm or less when the metal foil 52 is titanium or a titanium alloy. Further, when the metal foil 52 is beryllium or a beryllium alloy, a thickness thereof is preferably 8 μm or more and 16 μm or less. Moreover, when the metal foil 52 is stainless steel, a thickness thereof is preferably 3 μm or more and 6 μm or less.

Incidentally, in FIG. 2, illustration of a portion of the target material 51 disposed in the recessed portion 110 of the substrate 50 and a portion of the metal foil 52 covering the recessed portion 110 are omitted. However, when the target 5 is used, the target material 51 is filled in the entire recessed portion 110. Moreover, the entire recessed portion 110 is covered with the metal foil 52. While the target material 51 inside the recessed portion 110 and the metal foil 52 are preferably alloyed with and joined to each other, the target material 51 inside the recessed portion 110 and the metal foil 52 are provided in tight contact with each other with substantially not gap therebetween even when they are not joined to each other.

A metal layer made of a metallic material that can be alloyed with lithium may be provided on a surface of the metal foil 52 closer to the target material 51. Copper, a copper alloy, or the like can be used as the material of the metal layer. The metal layer may be formed on the surface of the metal foil 52 by sputtering, vacuum deposition, or the like. By providing the metal layer, the target material 51 and the metal layer can be alloyed with each other to be joined. Moreover, joinability between the substrate 50 and the metal foil 52 can be improved according to the material and the joining method. Furthermore, oxidation of the surface of the metal foil 52 can be prevented. Accordingly, deterioration of the joining strength due to the presence of an inclusion can be prevented.

When energy of the proton beam 6 is 1.88 MeV or more and 2.8 MeV or less and when the metal foil 52 is titanium or a titanium alloy and has a thickness of 4 μm or more, when the metal foil 52 is beryllium or a beryllium alloy and has a thickness of 8 μm or more, or when the metal foil 52 is stainless steel and has a thickness of 3 μm or more, a thickness of the target material 51 is preferably 250 μm or more. Further, a thickness of the target material 51 is preferably 350 μm or less. Note that a thickness of the target material 51 can be larger when energy of the proton beam 6 is 2.8 MeV or more.

Providing such a thickness of the target material 51 holds an end of the range of the proton beam 6 inside the target material 51. When the end of the range of the proton beam 6 is inside the target material 51, blistering may occur at an interface between the target material 51 and the substrate 50 or the like. Moreover, lithium hydride, which has low thermal conductivity, may be generated to lower efficiency of heat removal from the target material 51. However, it was confirmed that influence of hydrogen generated inside the target material 51 was small, in a durability test in which an irradiation time was 50 to 100 hours. On the other hand, when the end of the range of the proton beam 6 is behind the target material 51, detachment, an increase in thermal resistance, hydrogen embrittlement, and the like may occur due to blistering on the substrate 50 behind the target material 51.

When the end of the range of the proton beam 6 is inside the target material 51, influence of hydrogen generated inside the target material 51 is small and hydrogen is less likely to be generated in the substrate 50 behind of the target material 51. This feature eliminates a need for using tantalum having high blistering resistance, or the like as a backing substrate. The holding plate 50A and the back plate 50B can be made of a material with high thermal conductivity, such as copper or a copper alloy, leading to achievement of high thermal conductivity. Accordingly, efficiency of cooling the target material 51 and the like can be improved as compared to conventional configurations.

Generally, pure lithium is mainly used as a target material that causes a nuclear reaction represented by ⁷Li(p,n)⁷Be. However, using solid pure lithium as the target material entails a problem that it is difficult to provide the target material and the substrate, and the target material and the metal foil in tight contact with each other.

For example, in the case of filling molten lithium into the recessed portion provided in the substrate, it is not easy to fill the target material into the recessed portion with no gap since wettability of lithium on the materials of typical substrates is low. In such a case, there is a problem that an interstice is likely to be formed between the target material and the substrate after the melt solidifies. Such a problem can occur in case of the metal foil as well.

Further, if materials of the target material and the substrate material greatly differ therebetween in coefficient of thermal expansion, the target material and the substrate are likely to be detached from each other as they undergo thermal expansion and thermal shrinkage or receive external forces during the cooling after the filling of the melt and repeated use of the target. In such a case, there is a problem that a gap is likely to be formed between the target material and the substrate. Such a problem can occur in case of the metal foil as well.

Failing to keep the target material and the substrate in tight contact with each other as described above deteriorates heat transfer performance between the target material and the substrate, thereby decreasing efficiency of cooling the target material with the cooling mechanism provided on the substrate side at the time of irradiating the target with a proton beam. The decrease in the efficiency of cooling the target material can cause burnout, leakage of the melted target material, and so on. Further, failing to keep the target material and the substrate in tight contact with each other lowers mechanical joinability thereof and deteriorates durability thereof, resulting in a problem. Such problems can occur in case of the metal foil as well.

In contrast, in the neutron generation device target 5 according to the present embodiment, the target material 51 and the substrate 50, and also the target material 51 and the metal foil 52 are alloyed with each other to be joined. Accordingly, heat transfer and joinability between the members are improved. Since thermal resistance between the members is kept low, heat in the target material 51 and the metal foil 52 heated by heat input by the proton beam 6 can be efficiently removed to the cooling channels 130 in the substrate 50. Moreover, since joining strength between the members is enhanced, an occurrence of detachment and generation of a gap can be reduced.

In particular, in the case where the target material 51 and the substrate 50, and also the target material 51 and the metal foil 52 are alloyed with each other to be joined, hydrogen is less likely to concentrate at the interfaces even when hydrogen is generated as a result of incidence of the proton beam 6. This feature reduces not only an occurrence of detachment of the target material 51 and the like and generation of a gap due to thermal expansion and thermal shrinkage but also an occurrence of detachment and generation of a gap due to blistering. Further, since the target material 51 is alloyed with the surrounding members, a difference in coefficient of linear expansion between the members is small, thereby suppressing an impact of the thermal expansion and thermal shrinkage. Accordingly, a target 5 that is highly durable with respect to a use time thereof is obtained.

Incidentally, while the target material 51 may have a structure in which a part of lithium is alloyed or a structure in which entire lithium is alloyed, it is preferable that the target material has a structure in which a part of lithium is alloyed from the viewpoint of providing a sufficient amount of lithium to release neutrons. Moreover, a structure in which both portions closer to the substrate 50 and closer to the metal foil 52 are alloyed is more preferable than a structure in which only the portion closer to the substrate 50 is alloyed from the viewpoint of ensuring sufficient heat transfer and joinability between the members. The side near the metal foil 52 is preferably such that the target material 51 and a metal layer formed on a surface of the metal foil 52 are alloyed with each other.

A thickness of the metal layer formed on the surface of the metal foil 52 is not particularly limited as long as it is possible to reduce a loss of the proton beam 6 and ensure sufficient heat transfer and joinability between the members. The thickness of the metal layer is preferably 0.01 μm or more and more preferably 0.1 μm or more from the viewpoint of forming the metal layer with a stable film thickness. Further, the thickness of the metal layer is preferably 1 μm or less and more preferably 0.5 μm or less from the viewpoint of ensuring sufficient heat transfer and joinability between the members and also reducing a loss of the proton beam 6.

Next, a method of producing the neutron generation device target to be used as a neutron source for the neutron generation device 100 will be described.

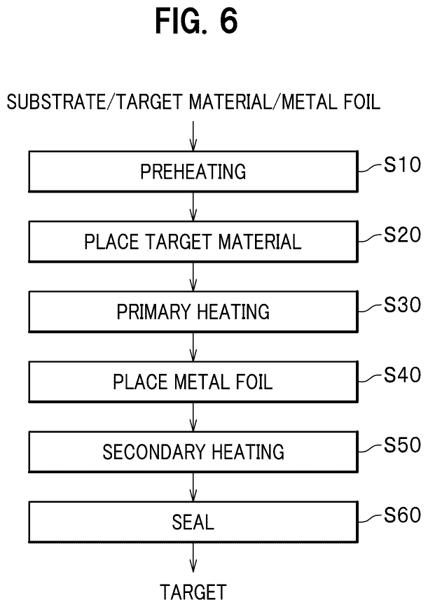

FIG. 6 is a diagram showing an example of the method of producing the neutron generation device target according to the embodiment of the present invention.

As shown in FIG. 6, the neutron generation device target 5 can be prepared by a production method including a preheating step S10, a target material placement step S20, a primary heating step S30, a metal foil placement step S40, a secondary heating step S50, and a sealing step S60. The following description will be given of a case of using the substrate 50 in which the holding plate 50A and the back plate 50B processed in advance are joined to each other as a substrate, and alloying both the side near the substrate 50 and the side near the metal foil 52 to be joined to each other.

In the preheating step S10, the substrate 50 to be used to prepare the target 5 is preheated to discharge impurity gases contained in the substrate 50. The substrate 50 to be used to prepare the target 5 may contain hydrogen which can cause hydrogen embrittlement and the like, oxygen, nitrogen, and moisture, which react with lithium, and so on. For this reason, these gases are removed by heating before placing the precursor of the target material 51 containing lithium in the recessed portion 110 of the substrate 50.

The preheating can be performed under a high vacuum condition of 10⁻⁵ Pa or more and 10⁻³ Pa or less or an ultra-high vacuum condition of 10⁻⁵ Pa or less. A temperature of the preheating is preferably such that the highest reachable temperature of the material is 200° C. or more. Time of the preheating is preferably 1 hour or more at the highest reachable temperature of the material.

In the preheating step S10, it is preferable to process not only the substrate 50 but also the metal foil 52 to release impurity gases contained in the metal foil 52. The preheating of the metal foil 52 can be performed under a similar condition to that of the preheating of the substrate 50. The substrate 50 and the metal foil 52 thus preheated are preferably handled under a high vacuum condition or an ultra-high vacuum condition in the subsequent steps.

In the target material placement step S20, a precursor of the target material 51 is placed in the recessed portion 110 provided in the substrate 50. The precursor of the target material 51 in solid form may be placed, or a liquid such as a melt of the precursor obtained by being melted via heating may be filled. As the precursor of the target material 51, a solid or liquid which, when solidifies, becomes equivalent in volume to the recessed portion 110 is preferably placed.

As the precursor of the target material 51, pure lithium metal may be placed or a lithium alloy may be placed. As the lithium alloy, a lithium-copper alloy, a lithium-aluminum alloy, a lithium-magnesium alloy, a lithium-zinc alloy, a multielement alloy containing three or more of these elements, or the like can be used. A foil of a metal such as aluminum, magnesium, or zinc may be interposed between the precursor of the target material 51 and the substrate 50 in advance, or a metal layer made of any of these metals may be formed between them in advance in order to adjust the wettability, the joinability, the coefficient of thermal expansion, and the like.

In the primary heating step S30, the substrate 50 with the precursor of the target material 51 placed in the recessed portion 110 is heated to melt the precursor of the target material 51 and then the precursor of the target material 51 is caused to solidify, so that the precursor of the target material 51 and the substrate 50 are alloyed with each other to be joined.

As the precursor of the target material 51 is heated to or above the melting point of lithium, impurity gases contained in the precursor, such as hydrogen, oxygen, and moisture, are released. Further, the heating also promotes diffusion of the atoms around the interface with the substrate 50. The alloying element contained in the substrate 50 gets diffused toward the precursor, thereby forming a concentration gradient in the thickness direction of the target 5. At a region near the interface with the substrate 50, the temperature is below the solidus of the multielement system, so that the lithium alloy starts solidifying. The alloying element gets diffused farther in the thickness direction and solidifies as the lithium alloy according to the cooling temperature. Thus, an alloy layer 51a, which is lithium and the alloying element alloyed with each other, is formed. Thereby, the formation of the alloy layer 51a can join the target material 51 and the substrate 50 to each other.

The primary heating can be performed under a high vacuum condition of 10⁻⁵ Pa or more and 10⁻³ Pa or less, or an ultra-high vacuum condition of 10⁻⁵ Pa or less. A temperature of the primary heating is preferably such that the highest reachable temperature of the material is a melting point of pure lithium (180° C.) or more, and more preferably such that the highest reachable temperature of the material is 230° C. or more. Time of the primary heating is preferably 1 hour or more in a state where the highest reachable temperature of the material is reached. The primary heating can be followed by cooling, such as natural air cooling or controlled cooling.

In the metal foil placement step S40, the metal foil 52 is placed on the substrate 50 to which the precursor of the target material 51 has been joined. The metal foil 52 is placed on a main surface of the substrate 50 with the recessed portion 110 accommodating the precursor of the target material 51 so as to cover a surface of the precursor of the target material 51 accommodated in the recessed portion 110.

The metal foil 52 is preferably such that it has a vertical width and a horizontal width which are larger than a vertical width and a horizontal width of the recessed portion 110, respectively, and impurity gases have been removed. A metal layer made of copper, a copper alloy, aluminum, magnesium, or zinc, which improves tightness of contact with the lithium, or the like may be formed on a surface of the metal foil 52 closer to the target material 51. It is preferable that the metal foil 52 is cleaned by removing an oxide film and the like on a surface thereof by physical etching or the like before the metal layer is formed, from the viewpoint of ensuring sufficient joining strength.

As a method of forming the metal layer, a vapor phase epitaxial method such as sputtering or vacuum deposition can be used. While the metal layer may be formed on both surfaces of the metal foil 52, it is preferably formed on one surface of the metal foil 52 in contact with the precursor of the target material 51. As the physical etching, ion milling, plasma etching, or the like with reverse sputtering using an inert gas, such as an argon gas, can be used.

In the secondary heating step S50, the substrate 50, the precursor of the target material 51, and the metal foil 52 are heated to alloy the precursor of the target material 51 and the metal foil 52 with each other to be joined. In the case where a metal layer has been formed on a surface of the metal foil 52, the precursor of the target material 51 and the metal layer can be alloyed with each other to be joined. Further, a main surface of the holding plate 50A on the front side holding the target material 51 and the metal layer can be joined to each other by interdiffusion of the atoms depending on the material of the metal layer.

As the precursor of the target material 51 is heated to or above the melting point of lithium, diffusion of the atoms around an interface with the metal foil 52 or the metal layer is promoted. Alloying elements contained in the metal foil 52 or the metal layer get diffused toward the precursor, thereby forming a concentration gradient in a thickness direction of the target 5. At a region near the interface with the metal foil 52 or the metal layer, a temperature thereof is below a solidus curve of the multielement system, so that the lithium alloy starts solidifying. The alloying elements get diffused farther in the thickness direction and solidifies as the lithium alloy responding to the cooling temperature. Thus, an alloy layer 51a made of lithium and the alloying elements alloyed with each other, is formed. Thereby, the formation of the alloy layer 51a can join the target material 51 and the metal foil 52 to each other via the alloy layer 51a.

The secondary heating can be performed under a high vacuum condition of 10⁻⁵ Pa or more and 10⁻³ Pa or less or an ultra-high vacuum condition of 10⁻⁵ Pa or less. A temperature of the secondary heating is preferably such that the highest reachable temperature of the material is a melting point of pure lithium (180° C.) or more, and more preferably such that the highest reachable temperature of the material is 230° C. or more. Time of the secondary heating is preferably 1 hour or more in a state where the highest reachable temperature of the material is reached. The secondary heating can be followed by cooling, such as natural air cooling or controlled cooling.

In the sealing step S60, a joint portion 150 is provided on a peripheral portion of the metal foil 52 to join the metal foil 52 and a surface of the substrate 50 in the side on which the proton beam 6 will be incident, i.e., a main surface of the holding plate 50A at a front side thereof, to each other. The joint portion 150 can be formed by various methods such as electron beam welding, laser welding, pressure welding, diffusion welding, and clinching. Providing the joint portion 150 prevents reactions between the lithium and the oxygen, nitrogen, moisture, and so on in the air and leakage of the target material 51 when it is melted. Providing the joint portion 150 also improves joining strength between the metal foil 52 and the substrate 50 side.

By going through the above steps, a lithium alloy material in which lithium is partly or entirely alloyed is formed as the target material 51 to be irradiated with a proton beam to generate neutrons. As a result, the neutron generation device target 5, in which the target material 51 and the substrate 50, and the target material 51 and the metal foil 52 are alloyed with each other to be joined, is obtained. The neutron generation device target 5 is obtained in a state where the metal foil 52 is joined to the surface of the target material 51 and also joined to an outer peripheral portion of the main surface of the holding plate 50A at the front side thereof via the joint portion 150. Note that the sealing step S60 can be omitted in the case of joining the main surface of the holding plate 50A at the front side holding the target material 51 and a metal layer provided on the metal foil 52 to each other by interdiffusion of the atoms by pressure welding or the like. In such a case, the metal foil 52 is obtained in a state where it is fused to both the surface of the target material 51 and the outer peripheral portion of the main surface of the holding plate 50A at the front side.

With the method of producing a neutron generation device target according to the embodiment described above, a lithium alloy material in which lithium is partly or entirely alloyed is formed as the target material 51 to be caused to generate neutrons. Hence, the target material 51 and the substrate 50, and the target material 51 and the metal foil 52 are formed in tight contact with each other. Alloying the target material 51 enhances tightness of contact between the metals, thus lowering thermal resistance thereof, and also reduces a difference in coefficient of thermal expansion. Accordingly, heat transfer and joinability of the target material 51 with the substrate 50 and the metal foil 52 are improved.

In particular, with the method of producing a neutron generation device target according to the embodiment described above, solid metallic lithium or a solid lithium alloy can be used as a precursor of the target material 51. Thus, operations under a depressurized atmosphere where a degree of vacuum is high can be performed more easily than in the case of using lithium melt. Since wettability of lithium and fillability of lithium melt do not impose a great impact, the substrate can be selected by mainly considering the heat transfer and joinability. Moreover, joining the metal foil 52 through interdiffusion of atoms by pressure welding or the like and with the joint portion 150 enhances joining strength between the metal foil 52 and the substrate 50 and therefore ensures reliable sealing of the target material 51.

Note that, in the case of alloying only a portion of the target material 51 closer to the substrate 50 (see FIG. 5B), the precursor of the target material 51 and the substrate 50 are alloyed with each other to be joined, and then the metal foil 52 is placed on the substrate 50 to which the precursor of the target material 51 has been joined. The metal foil 52 and the substrate 50 can then be joined by performing the sealing step S60 without performing the secondary heating step S50.

Further, in the case of forming a structure in which lithium is entirely alloyed (see FIG. 5C), the target material 51 and the substrate or the target material 51 and the metal foil 52 can be joined to each other by adjusting an amount of the alloying element to be added to the precursor of the target material 51, a concentration of the alloying element contained in the substrate 50, the metal foil 52, the metal layer, or the like, temperatures and time of the primary heating and secondary heating, and so on.

Further, in the steps described above, the primary heating step S30 and the secondary heating step S50 are performed, but a single-stage heating step may be performed. In the case of performing a single-stage heating step, the precursor of the target material 51 is placed on the substrate 50 and the metal foil 52 is placed on the substrate 50 so as to cover the precursor of the target material 51. Then, the substrate 50, the target material 51, and the metal foil 52 are heated. A metal layer may be formed on a surface of the metal foil 52 in advance. With the single-stage heating step too, after the precursor of the target material 51 is melted by the heating, while the precursor of the target material 51 is caused to solidify, it is possible to alloy the precursor of the target material 51 and the substrate 50, and the precursor of the target material 51 and the metal foil 52 or the metal layer with each other to be joined together.

While the present invention has been described above, the present invention is not limited to the foregoing embodiment, and various changes can be made without departing from the gist of the present invention. For example, the present invention is not necessarily limited to one consisting of all of the components included in the foregoing embodiment. It is possible to replace some of the components in an embodiment to another component(s), add some of the components in an embodiment to another embodiment, and omit some of the components in an embodiment.

For example, while the substrate 50 in the target 5 described above is formed by stacking the holding plate 50A and the back plate 50B to each other, thicknesses of the members to be stacked to each other and the number of the members are not particularly limited. The substrate 50 may be formed of a single member or three or more members as long as the cooling channels 130 can be formed appropriately and the substrate 50 can be mounted to the target holder appropriately.

Further, while in the holding plate 50A and the back plate 50B in the target 5 described above are provided in a rectangular shape in a plan view and have the same length and width, the lengths and widths are not particularly limited. For example, it is possible to employ a configuration in which the substrate 50 is formed of a single sheet of a material, and a portion of the target holder is used as a back plate 50B to close one side of wall surfaces of the cooling channels 130.

### EXAMPLES

Hereinafter, the present invention will be specifically described by showing examples, but the technical scope of the present invention is not limited to these.

Results of a heat load test by electron beam irradiation, a heat load test by proton beam irradiation, and an elemental analysis by laser-induced breakdown spectroscopy (LIBS) analysis on prepared neutron generation device targets will be described.

As the neutron generation device targets, rectangular targets in which a target material and a substrate, and the target material and a metal foil were joined to each other as shown in FIG. 2 were prepared. The neutron generation device targets were prepared by placing a precursor of the target material in a recessed portion provided in the substrate, placing the metal foil on the substrate such that the metal foil covered the precursor of the target material, and then performing heat treatment on the entire body.

The precursor of the target material was pure lithium with a Li content of 99.8% or more. The substrate was oxygen-free copper with a Cu content of 99.96% or more. The metal foil was pure titanium with a Ti content of 99.9% or more. The target measured 110 mm×110 mm×10.5 mm. The recessed portion measured 60 mm×60 mm×0.5 mm.

For one target, a metal foil with a Cu coating was used. The target using the metal foil with a Cu coating was prepared by forming a metal layer on one surface of the metal foil in advance by Cu vapor deposition and then alloying the target material and the metal layer with each other. The metal layer was prepared by removing a passivation film on the one surface of the metal foil by reverse sputtering of the surface with an argon gas, and then vapor-depositing pure copper.

For the other target, a metal foil with no Cu coating was used. The target using the metal foil with no Cu coating was prepared by joining a metal foil to the substrate on which the target material was placed without pure copper vapor-deposited on a surface of the metal foil.

The heat load test by electron beam irradiation was performed on the target using the metal foil with a Cu coating and the target using the metal foil with no Cu coating with use of an electron gun device. An electron beam was irradiated toward a center of each target material covered with the metal foil in a normal direction of the target material. A region to be irradiated with the electron beam was adjusted such that a horizontal half width was 24.4 mm, a vertical half width was 15.4 mm, and an irradiation area was approximately 1190 mm².

The heat load test by proton beam irradiation was performed on the target using the metal foil with a Cu coating with use of an accelerator. A proton beam was irradiated toward a center of the target material covered with the metal foil in a normal direction of the target material. Energy of the proton beam was 1.8 MeV. This condition prevents the target material from generating neutrons. A current value of the proton beam was 3.5 mA. An irradiation area of the proton beam was approximately 700 mm². Density of heat input into the target in this state is calculated to be approximately 9.0 MW/m².

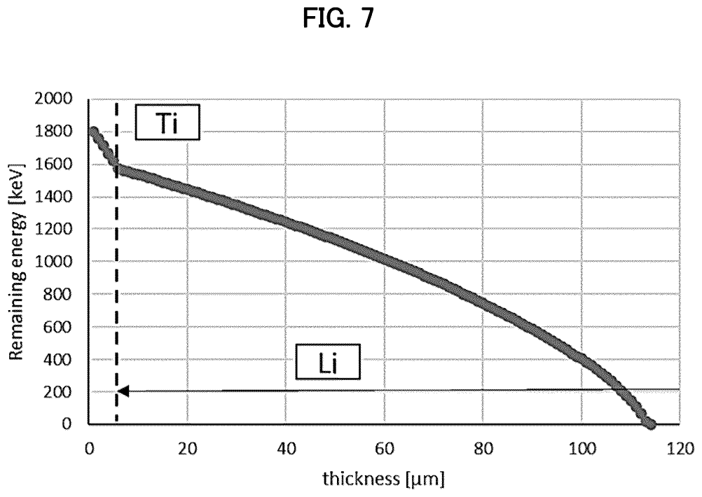

FIG. 7 is a diagram showing an energy loss of a proton beam in a neutron generation device target. In FIG. 7, the horizontal axis represents a thickness (i.e., depth) [μm] from the surface of the metal foil of the target while the vertical axis represents remaining energy [keV] of the proton beam.

As shown in FIG. 7, in a heat resistance test with proton beam irradiation, such a condition that energy of the irradiated proton beam would be lost inside the target material was employed. It was such a condition that a proton beam of approximately 1.6 MeV having passed through a Ti metal foil would be incident on the target material.

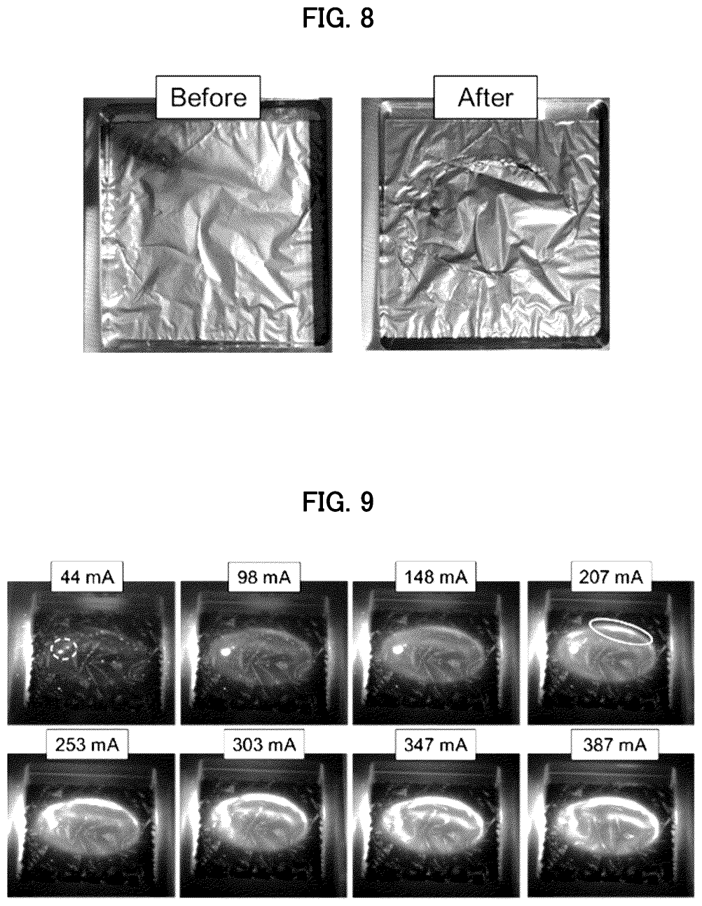

FIG. 8 is a set of photographic images showing an exterior of the neutron generation device target using the metal foil with a Cu coating before being irradiated with an electron beam and after being irradiated with the electron beam. A left image in FIG. 8 represents an exterior of the Ti foil before the electron beam irradiation, while a right image in FIG. 8 represents an exterior of the Ti foil after the electron beam irradiation.

FIG. 9 is a set of infrared photographic images of the neutron generation device target using the metal foil with a Cu coating during the electron beam irradiation. Each image represents a thermal image of a surface of the target at a current value in the process of increasing the current value of the electron beam from 40 mA to approximately 380 mA or more.

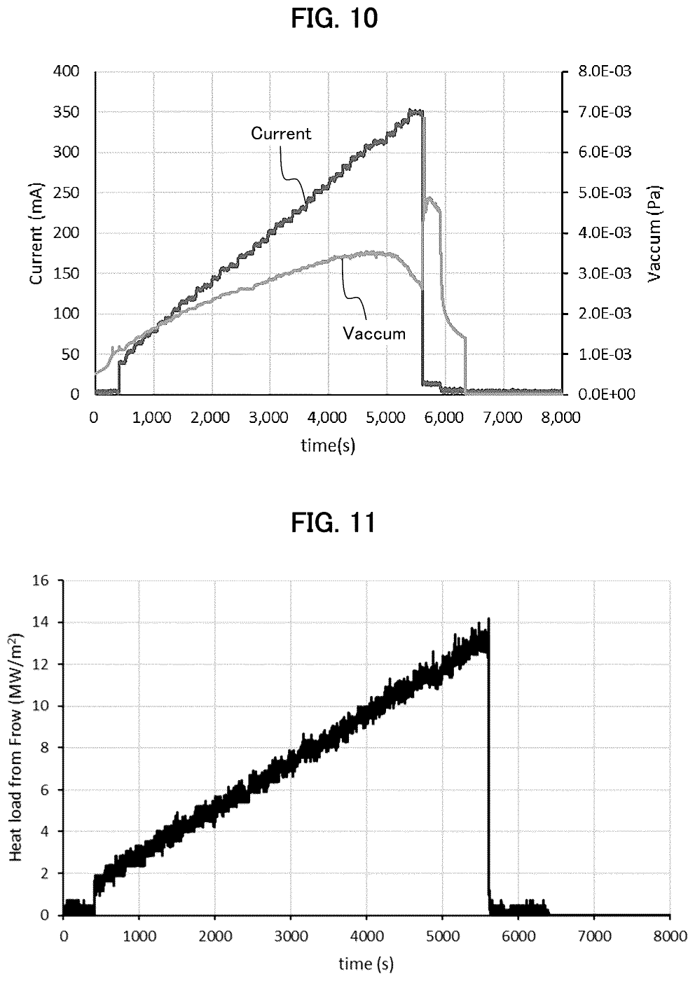

FIG. 10 is a diagram showing a temporal change in current value and a temporal change in a degree of vacuum during the electron beam irradiation of the neutron generation device target using the metal foil with a Cu coating. In FIG. 10, the horizontal axis represents time [s], the left vertical axis represents a beam current value [mA] of the electron beam irradiated to the target, and the right vertical axis represents a degree of vacuum [Pa] in the atmosphere in which the target was placed.

As shown in FIG. 10, as the electron beam irradiation continued, the degree of vacuum slightly recovered at around 5000 s. Then, at around 5500 s, the degree of vacuum abruptly deteriorated and the beam current value abruptly decreased as well. The recovery of the degree of vacuum is presumed to have occurred due to evaporation of Ti in the metal foil, resulting in a getter effect with the Ti. The deterioration of the degree of vacuum and the decrease of the beam current value are presumed to have occurred due to burnout of the Ti foil by intense heat, resulting in release of outgases contained in voids and the like in the Li layer.

FIG. 11 is a diagram showing a temporal change in density of heat input during the electron beam irradiation of the neutron generation device target using the metal foil with a Cu coating. In FIG. 11, the horizontal axis represents time [s], while the vertical axis represents density [Mw/m²] of heat input into the target by the electron beam.

As shown in FIG. 11, the density of heat input into the target was approximately 11 MW/m² at around 5000 s, at which the degree of vacuum slightly recovered (see FIG. 10). Before the degree of vacuum slightly recovered, there was substantially no burnout or evaporation of the Ti foil. Thus, it can be said that the target was sound. Hence, resistance of the neutron generation device target using the metal foil with a Cu coating against a heat load can be considered to have improved to approximately 11 MW/m².

FIG. 12 is a set of photographic images showing an exterior of the neutron generation device target using the metal foil with no Cu coating before being irradiated with an electron beam and after being irradiated with the electron beam. The left image in FIG. 12 represents an exterior of the Ti foil before the electron beam irradiation, while the right image in FIG. 12 represents an exterior of the Ti foil after the electron beam irradiation.

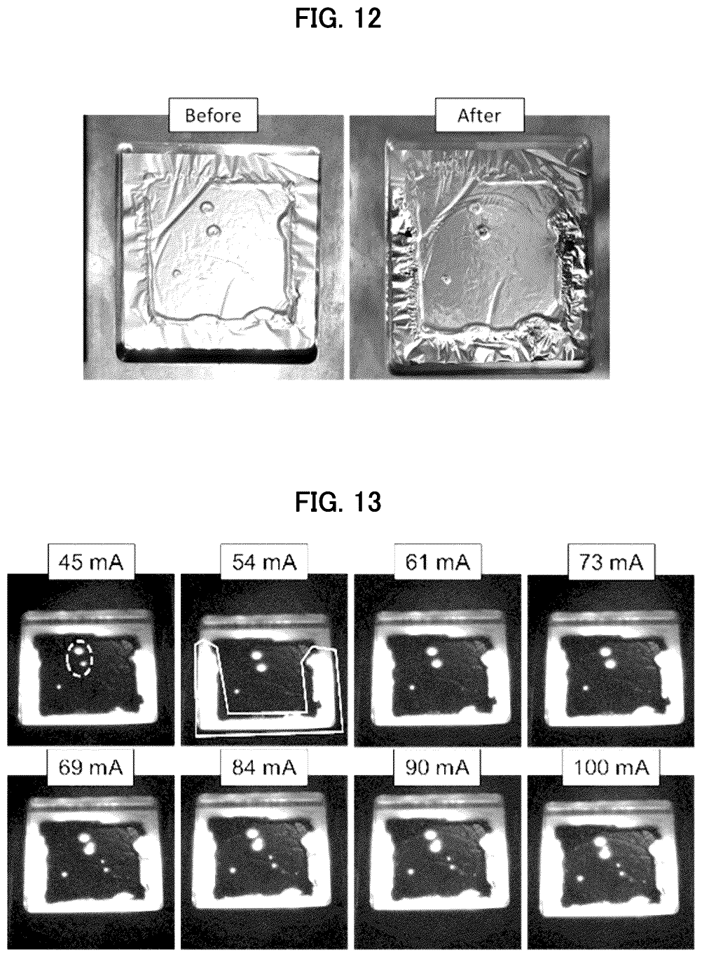

FIG. 13 is a set of infrared photographic images of the neutron generation device target using the metal foil with no Cu coating during the electron beam irradiation. Each image represents a thermal image of a surface of the target at a current value in the process of increasing the current value of the electron beam from 40 mA to approximately 100 mA or more.

As shown in FIG. 13, circular bright spots appeared in a region irradiated with the electron beam. The circular bright spots are presumed to represent voids in the Li layer. At the regions with the voids, the target material and the Ti foil were not joined and interstices were formed, thereby lowering the heat removal efficiency, so that the temperature was high. Areas of the voids became larger with an increase in a current value. Incidentally, at a peripheral portion of the target material, the target material and the Ti foil were not joined, and the temperature was high due to the low heat removal efficiency.

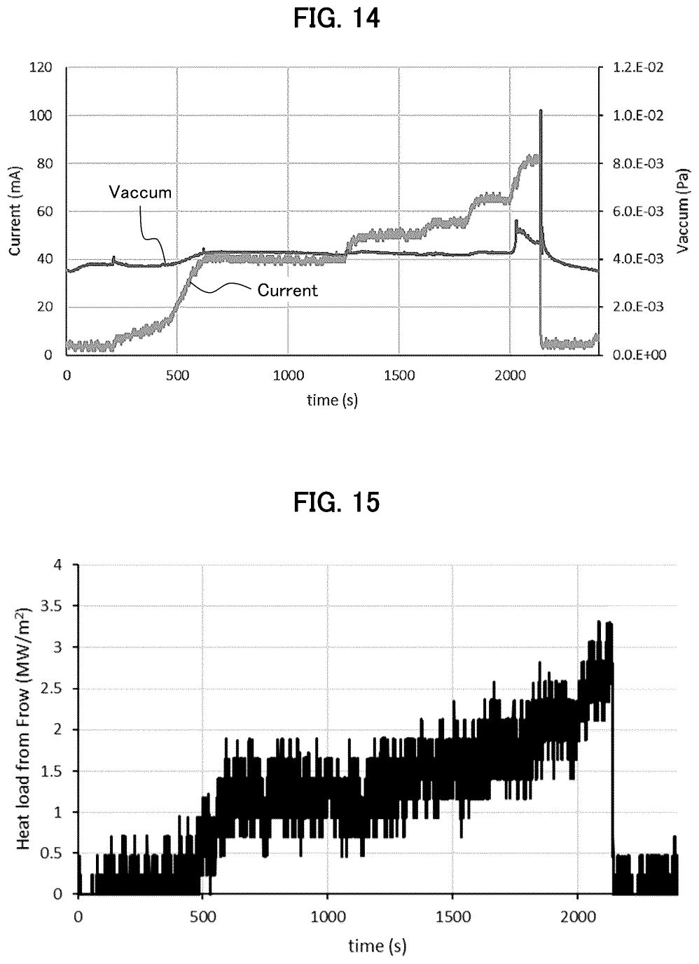

FIG. 14 is a diagram showing a temporal change in a current value and a temporal change in a degree of vacuum during the electron beam irradiation of the neutron generation device target using the metal foil with no Cu coating. In FIG. 14, the horizontal axis represents time [s], the left vertical axis represents a beam current value [mA] of the electron beam irradiated to the target, and the right vertical axis represents a degree of vacuum [Pa] in the atmosphere in which the target was placed.

As shown in FIG. 14, as the electron beam irradiation continued, a degree of vacuum rose at around 2000 s. Then, the degree of vacuum abruptly deteriorated and the beam current value abruptly decreased as well. The deterioration of the degree of vacuum and the decrease of the beam current value are presumed to have occurred due to burnout of the Ti foil by intense heat, resulting in release of outgases contained in voids and the like in the Li layer.

FIG. 15 is a diagram showing a temporal change in density of heat input during the electron beam irradiation of the neutron generation device target using the metal foil with no Cu coating. In FIG. 15, the horizontal axis represents time [s], while the vertical axis represents density [Mw/m²] of heat input into the target by the electron beam.

As shown in FIG. 15, density of heat input into the target was approximately 2 MW/m² at around 2000 s, at which a degree of vacuum rose (see FIG. 14). Before the degree of vacuum slightly rose, there was substantially no burnout or evaporation of the Ti foil. Thus, it can be said that the target was sound. Hence, the neutron generation device target using the metal foil with no Cu coating can be considered to have low resistance against a heat load as compared to the target using the metal foil with a Cu coating, which withstood a heat input density of approximately 11 MW/m².

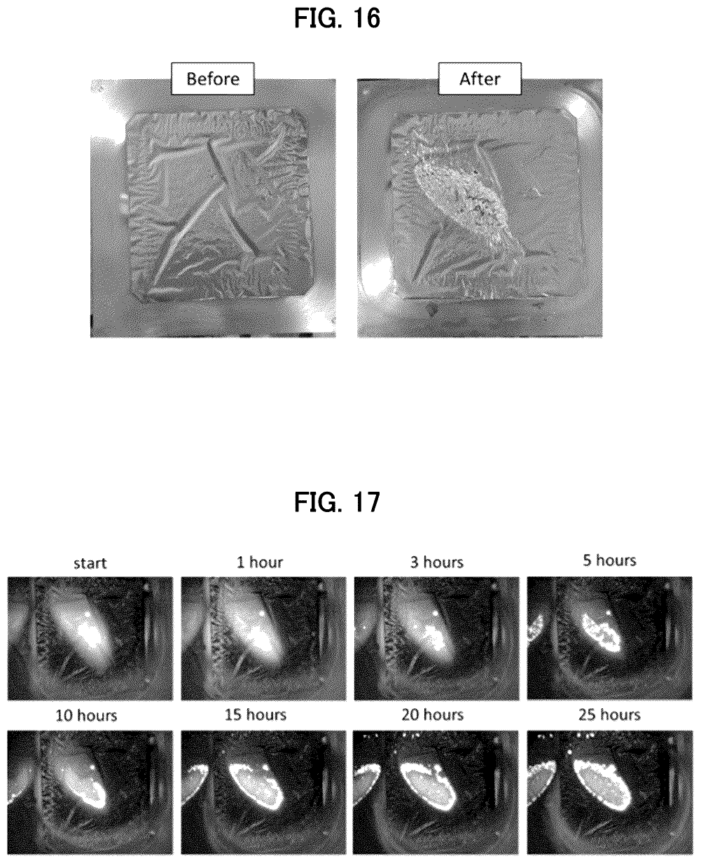

FIG. 16 is a set of photographic images showing an exterior of the neutron generation device target using the metal foil with a Cu coating before being irradiated with a proton beam and after being irradiated with the proton beam. The left image in FIG. 16 represents an exterior of the Ti foil before the proton beam irradiation, while the right image in FIG. 16 represents an exterior of the Ti foil after the proton beam irradiation.

As shown in FIG. 16, discoloration of the Ti foil was observed under this irradiation condition, but no severe burnout or the like was observed.

FIG. 17 is a set of infrared photographic images of the neutron generation device target using the metal foil with a Cu coating during the proton beam irradiation. Each image represents a thermal image of a surface of the target at each irradiation time in the proton beam irradiation.

As shown in FIG. 17, an irregular shaped bright spot appeared in a region irradiated with the proton beam at the initial stage of the irradiation. The irregular shaped bright spot is presumed to represent a void in the Li layer. As the irradiation time passed, regions with voids increased and moved. A void was present at a center portion of the irradiation region at the initial stage of the irradiation, but uniform low temperatures were observed as the irradiation time passed. It is possible that, at the regions with voids, temperatures of the target material and the Ti foil rose, thus allowing the alloying of Li and Cu to progress. It can be considered that the alloying improved tightness of contact between the target material and the Ti foil and thus improved heat removal efficiency, leading to the low temperatures.

According to the heat load test by electron beam irradiation described above, using the metal foil with a Cu coating improved performance of heat removal by the cooling channels as compared to the case of using the metal foil with no Cu coating. Therefore, it can be said that using the metal foil with a Cu coating provide such high heat load resistance as to withstand an approximately five times higher heat input density, that is, approximately 11 MW/m². Further, according to the heat load test by proton beam irradiation described above, it can be said that such high durability as to withstand irradiation with a proton beam for 26 hours under conditions of energy of the proton beam being 1.8 MeV, a current value of the proton beam being 3.5 mA, and density of heat input into the target being approximately 9.0 MW/m² is obtained.

### REFERENCE SIGNS LIST

100 neutron generation device
1 proton beam generation device
1a ion source
1b accelerator
2 neutron deceleration irradiation device
2a moderator
2b collimator
3 irradiation object
4 channel
5 target
6 proton beam
7 focusing lens
9 neutron beam
50 substrate
50A holding plate
50B back plate
51 target material
51a alloy layer
52 metal foil
56 rib
110 recessed portion
120 straight groove
130 cooling channel

1. A target for a neutron generation device for generating neutrons by a ⁷Li(p,n)⁷Be reaction, the target comprising:
a target material to generate neutrons in response to being irradiated with a proton beam;
a substrate including a recessed portion for holding the target material; and
a metal foil which seals the target material held in the recessed portion, wherein
the target material is a lithium alloy material containing lithium partly or entirely alloyed, and
the target material and the substrate are alloyed with each other and joined together.

2. The target for a neutron generation device according to claim 1, wherein the target material and the substrate, and the target material and the metal foil are alloyed with each other and joined together.

3. The target for a neutron generation device according to claim 1, wherein the target material is a lithium alloy material containing lithium and at least one of copper, aluminum, magnesium, and zinc.

4. The target for a neutron generation device according to claim 1, wherein
the metal foil is titanium, a titanium alloy, beryllium, a beryllium alloy, or stainless steel, and
the substrate is copper or a copper alloy.

5. The target for a neutron generation device according to claim 1, wherein a joint portion is provided which joins the metal foil and a surface of the substrate on which the proton beam is to be incident to each other.

6. The target for a neutron generation device according to claim 1, wherein
energy of the proton beam is 1.88 MeV or more and 2.8 MeV or less,
the metal foil is titanium, a titanium alloy, beryllium, a beryllium alloy, or stainless steel,
a thickness of the metal foil is 4 μm or more when the metal foil is titanium or a titanium alloy, 8 μm or more when the metal foil is beryllium or a beryllium alloy, and 3 μm or more when the metal foil is stainless steel, and
a thickness of the target material is 250 μm or more.

7. A method of producing a target for a neutron generation device for generating neutrons by a ⁷Li(p,n)⁷Be reaction,
the target including a target material to generate neutrons in response to being irradiated with a proton beam, a substrate including a recessed portion for holding the target material, and a metal foil which seals the target material held in the recessed portion,
the method comprising:
placing a precursor of the target material made of metallic lithium or a lithium alloy in the recessed portion provided in the substrate;
alloying the precursor and the substrate with each other to be joined together by heating the substrate and the precursor to melt the precursor and then solidifying the precursor; and
joining a metal foil onto the substrate to which the precursor has been joined.

8. The method of producing a target for a neutron generation device according to claim 7, further comprising:
placing a metal foil on the substrate to which the precursor has been joined after alloying the precursor and the substrate with each other to be joined together; and
alloying the precursor and the metal foil with each other to be joined together by heating the substrate, the precursor and the metal foil.

9. The method of producing a target for a neutron generation device according to claim 8, further comprising:
preheating the substrate to release a gas in the substrate before placing the precursor in the recessed portion; and
preheating the metal foil to release a gas in the metal foil before placing the metal foil on the substrate.

10. The method of producing a target for a neutron generation device according to claim 8, further comprising
joining the metal foil and a surface of the substrate on which the proton beam is to be incident to each other after alloying the precursor and the metal foil with each other to be joined together.

11. The method of producing a target for a neutron generation device according to claim 7, further comprising:
forming a metal layer on a surface of the metal foil and alloying the precursor and the metal layer with each other to be joined before joining the metal foil onto the substrate.

12. The method of producing a target for a neutron generation device according to claim 11, wherein
the metal foil is titanium, a titanium alloy, beryllium, a beryllium alloy, or stainless steel,
the metal layer is made of copper, and
the method further comprises cleaning the metal foil by physical etching before forming the metal layer.
